(19) United States
(12) Patent Application Publication
(10) Pub. No.: US 2008/0029455 A1
(43) Pub. Date: Feb. 7, 2008

(54) Separation Methods

(75) Inventor: Daniel Brian Jones, Cambridgeshire (GB)

Correspondence Address: WOLF GREENFIELD & SACKS, P.C., 600 ATLANTIC AVENUE, BOSTON, MA, 02210-2206, US

(73) Assignee: Novexin Limited, Cambridgeshire (GB)

(21) Appl. No.: 10/578,526

(22) PCT Filed: Nov. 8, 2004
(86) PCT No.: PCT/GB04/50024

§ 371 Date: Apr. 9, 2007

(30) Foreign Application Priority Data
Nov. 7, 2003 (GB) 0326064.3

(51) Int. Cl. C07K 1/20

(52) U.S. Cl. 210/638

(57) ABSTRACT

A method for chromatographic separation of a molecule, wherein a mobile phase and a charged stationary phase are present and a charged amphipathic sugar polymer(s) is employed to modify the hydrophobic interaction between the molecule and said charged stationary phase.

The invention relates to separation methods for separation, purification and isolation of molecules, based on hydrophobic interaction chromatography and on mixed-mode hydrophobic interaction/ion exchange chromatography.

### BACKGROUND TO THE INVENTION

Liquid chromatography is a commonly used protein purification technique due to its high capacity and selectivity. The technique depends upon the interactions of macromolecules in solution with a packed bed of a chromatography matrix. Interactions between the macromolecules and chromatography matrix may be based on size, charge, hydrophobicity or a more specific type of interaction (e.g. antibody—antigen binding). This leads to four broad classes of chromatography; size exclusion (gel permeation), ion exchange, hydrophobic interaction and affinity.

Ion exchange is the most commonly used method for the preparative scale purification of proteins, polypeptides, nucleic acids and other charged biomolecules (Bonnerjera et al., 1986, Bio/Technology; Freitag and Horvath, Advances in Biochemical Engineering/Biotechnology, 1995.). The advantages of ion exchange chromatography over alternative methods are its widespread applicability, relative simplicity, high capacity and the relatively low cost of ion exchange matrices.

Separation of molecules during ion exchange chromatography depends on the reversible adsorption (non-covalent association) of charged molecules with a charged chromatography matrix. The molecule(s) of interest may carry the opposite charge to the chromatography matrix under the selected experimental conditions. The first stage in such purification is binding of the target molecule to the matrix through electrostatic Interactions. This is followed by an elution step where the molecule is released from the matrix. Release can be achieved by altering the solution pH or ionic strength to create conditions that are unfavourable for molecule—matrix interactions, so that the association is disrupted and the molecule can be eluted from the chromatography matrix.

Elution is typically performed by introducing a gradually increasing salt concentration to the column. Weakly bound molecules are eluted first, followed by more strongly bound substances. Such purifications can also be performed under conditions where the target molecule does not bind to the chromatography matrix, instead it passes through the matrix, the contaminants being removed through binding to the column.

Both anion, exchange (positively charged) and cation exchange (negatively charged) matrices are commercially available. Such matrices can have varying degrees of ionisation, depending on pH. Strong ion exchangers are completely ionised over a wide pH range, whilst the ionisation and hence binding capacity, of weak ion exchangers is more easily influenced by changes in buffer pH. Typical functional groups on the surface of ion exchange matrices include quarternary ammonium (strong anion exchanger), sulfonic acid (strong cation exchanger), diethylaminoethyl (weak anion exchanger) and carboxymethyl (weak cation exchanger).

Cyclodextrin and charged cyclodextrin derivatives have been used in capillary electrophoresis as selectivity and resolution enhancers. During electrophoresis, protein migration is driven by the application of an electric field. Many types of detergents and certain types of cyclodextrins have been used to alter the behaviour of various proteins during electrophoresis (Weinberger, Practical Capillary Electrophoresis, 2000; Rathore and Horvath, Electrophoresis, 1998). Covalently immobilised cyclodextrin has been used as a chiral chromatography matrix for enanfiomer separation, these separations are based on the stereoselectivity for the cyclodextrin cavity and do not involve a charged stationary phase such as an ion exchange chromatography matrix.

Inulin beads have been used in chromatography, but these have not been charged and have not been used with a charged stationary phase. To date, charged inulins have not been reported to be useful as chromatographic separation aids. Inulins are D-fructans, generally consisting of chains of polyfructose in which the fructose units are connected to each other mostly or exclusively by β-(2-1) linkages. Inulin occurs in nature, in general, as a polydisperse mixture of polyfructose chains most of which are ending in one glucosyl unit. Inulin can be obtained from bacterial syntheses, extracted from plants or can be made in vitro by bnzymatic synthesis starting from sucrose. Inulin produced by bacteria is more branched than inulln from plant origin and has commonly a higher molecular weight (ranging from about 2,000 up to about 20,000,000), whereas inulin from plant origin is generally composed of linear or slightly branched polyfructose chains or mixtures thereof with a molecular weight commonly ranging from about 600 to about 20,000.

Inulin can be represented, depending from the terminal carbohydrate unit, by the general formulae GF.n or F.n, wherein G represents a glucosyl unit, F a fructosyl unit, and n is an integer representing the number of fructosyl units linked to each other in the carbohydrate chain. The number of saccharide units (fructose and glucose units) in one inulin molecule, i.e. the values n+1 and n in the formulae above, are referred to as the degree of polymerisation, represented by (DP). Often, the parameter (number) average degree of polymerisation, represented by (DP), is used too, which is the value corresponding to the total number of saccharide units (G and F units) in a given inulin composition divided by the total number of inulin molecules present in said inulin composition, without taking into account the possibly present monosaccharides glucose (G) and fructose (F), and the disaccharide sucrose (GF). The average degree of polymerisation (DP) can be determined, for example, by the method described by L. De Leenheer (Starch, 46 (5), 193-196, (1994), and in “Carbohydrates as Organic Raw Materials”, Vol. III, p. 67-92, (1996)).

Inulin is commonly prepared from plant sources, mainly from roots of Chicory (Cichodum intybus) and from tubers of Jerusalem artichoke (Helianthus tuberosus), in which inulin can be present in concentrations of about 10 to 20% w/w on fresh plant material. Inulin from plant origin is usually a polydisperse mixture of linear and slightly branched polysaccharide chains with a degree of polymerisation (DP) ranging from 2 to about 100. In accordance with known techniques, inulin can be readily extracted from said plant parts, purified and optionally fractionated to remove impurities, mono- and disaccharides and undesired oligosaccharides, in order to provide various grades of inulin, as e.g. described in EP 0 769 026 and EP 0 670 850.

Inulin is commercially available, typically with a (DP) ranging from about 6 to about 40. Inulin from chicory is for example available as RAFTILINE® from ORAFTI, (Tienen, Belgium) in various grades. Typical RAFTILINE® grades include RAFTILINE® ST (with a (DP) of about 10 and containing in total up to about 8% by weight glucose, fructose and sucrose), RAFTILINE® LS (with a (DP) of about 10 but containing in total less than 1% by weight glucose, fructose and sucrose), and RAFTILINE®. RTM. HP (with a (DP) of at least 23, commonly with a (DP) of about 25, and virtually free of glucose, fructose and sucrose).

Inulins with a lower degree of polymerisation, usually defined as a (DP) <10, are commonly named inulo-oligosaccharides, fructo-oligosaccharides or oligofructose. Oligofructose can be conventionally obtained by partial (preferably enzymatic) hydrolysis of inulin and can also be obtained by enzymatic in vitro synthesis from sucrose according to techniques which are well-known in the art. Several grades of oligofructose are commercially available, for example as RAFTILOSE® from Orafti, (Tienen, Belgium), e.g. RAFTILOSE® P95 with a mean content of about 95% by weight of oligofructose with a degree of polymerisation (DP) ranging from 2 to 7 and containing about 5% by weight in total of glucose, fructose and sucrose.

Various inulin derivatives and methods for the preparation of inulin derivatives are described in U.S. Pat. No. 6,534,647 (Stevens et al), the entire contents of which are incorporated herein by reference.

Inulins, both underivatised (for example Inutec®PN25 (uncharged)) and derivatised with hydrophobic alkyl chains on the polyfructose backbone (for example Inutec®SP1 (SP1) (uncharged)) are commercially available from Orafti (Tienen, Belgium).

### DISCLOSURE OF THE INVENTION

The present invention provides a method for chromatographic separation of a molecule, wherein a mobile phase and charged stationary phase are present and a charged amphipathic sugar polymer(s) is employed to modify the hydrophobic interaction between the molecule and said charged stationary phase.

In methods of the invention, charged amphipathic sugar polymers alter the level of interaction between the molecules, in particular charged molecules and a charged stationary phase such as an ion exchange resin. The separation methods of the invention are useful for separation, purification and isolation of molecules based on hydrophobic interaction chromatography or on mixed-mode hydrophobic interaction/ion exchange chromatography.

The present invention provides a method for separating a molecule from a solution comprising the molecule and further components by hydrophobic interaction chromatography comprising applying the solution comprising the molecule to a charged stationary phase, and eluting the molecule from the stationary phase in a mobile phase, characterised in that:
(a) the charged stationary phase is non-covalenty associated with a charged amphipathic sugar polymer(s), or the stationary phase comprises a charged amphipathic sugar polymer, and/or
(b) the molecule is non-covalently associated with a charged amphipathic sugar polymer(s).

In some methods of the invention, the pH chosen for the solution comprising the molecule and/or for the mobile phase is below the pI of the molecule and thus the molecule carries a net positive charge. Alternatively, the pH of the solution and/or the mobile phase can be chosen so that it is above the pI of the molecule and thus the molecule carries a net negative charge. The pI is the pH at the isoelectric point. The solution comprising the molecule may be prepared by dissolving the molecule in the mobile phase running buffer or by diluting the molecule in the mobile phase running buffer.

The present invention provides a method for separating a positively charged molecule from a solution comprising the molecule and further components by hydrophobic interaction chromatography comprising applying a solution comprising the molecule to a positively charged stationary phase (e.g. an anion exchanger) which is non-covalently associated with a negatively charged amphipathic sugar polymer(s) (e.g. sulfated beta-cyclodextrin or sulfonated inulin), and eluting the molecule from the stationary phase in a mobile phase,

The present invention further provides a method for separating a negatively charged molecule from a solution comprising the molecule and further components by hydrophobic interaction chromatography comprising applying a solution comprising the molecule to a negatively charged stationary phase (e.g. a cation exchanger) which is non-covalently associated with a positively charged amphipathic sugar polymer(s), and eluting the molecule from the stationary phase in a mobile phase.

The present invention also provides a method for separating a positively charged molecule from a solution comprising the molecule and further components by mixed mode hydrophobic interaction/ion exchange chromatography comprising applying a solution comprising the molecule to a negatively charged stationary phase (e.g. a cation exchanger) which is non-covalently associated with a positively charged amphipathic sugar polymer(s) (e.g. amino beta-cyclodextrin or aminopropyl inulin), and eluting the molecule from the stationary phase in a mobile phase.

The present invention further provides a method for separating a negatively charged molecule from a solution comprising the molecule and further components by mixed mode hydrophobic interaction/ion exchange chromatography comprising applying a solution comprising the molecule to a positively charged stationary phase (e.g. an anion exchanger) which is non-covalently associated with a negatively charged amphipathic sugar polymer(s) (e.g. a carboxymethyl cyclodextrin or a carboxy methyl inulin), and eluting the molecule from the stationary phase in a mobile phase.

The mobile phase employed in methods of the invention may comprise an amphipathic sugar polymer(s), i.e. an amphipathic sugar polymer or a mixture of amphipathic sugar polymers. The mobile phase may comprise a charged amphipathic polymer(s). In methods where the amphipathic sugar polymer(s) are included in the solution and/or mobile phase (e,g. running buffer) the amphipathic sugar polymer or mixture of amphipathic sugar polymers is suitably present at from 0.01 to 50 mg/ml, preferably from 0.01 to 20 mg/mI, more preferably from 0.1 to 10 mg/ml.

The invention provides a method for separating a positively charged molecule from a solution comprising the molecule and further components by mixed mode hydrophobic interaction/ion exchange chromatography comprising applying a solution comprising the molecule to a negatively charged stationary phase (e.g. a cation exchanger) to the stationary phase, and eluting the molecule from the stationary phase in a mobile phase comprising a negatively charged amphipathic polymer(s) (e.g. a carboxy methyl cyclodextrin or carboxy methyl inulin).

The invention further provides a method for separating a negatively charged molecule from a solution comprising the molecule and further components by mixed mode hydrophobic interaction/ion exchange chromatography comprising applying a solution comprising the molecule to a positively charged stationary phase (e.g. an anion exchanger), and eluting the molecule from the stationary phase in a mobile phase comprising a positively charged amphipathic sugar polymer(s).

In an aspect of a method of the invention, the charged stationary phase may comprise or consist of a charged amphipathic sugar polymer(s).

Thus the invention provides a method for separating a molecule from a solution comprising a molecule and further components by hydrophobic interaction chromatography comprising applying the solution comprising the molecule to a charged stationary phase, and eluting the molecule from the stationary phase in a mobile phase, characterised in that the stationary phase comprises a charged amphipathic sugar polymer(s).

Also provided is a method for separating a molecule from a solution comprising a charged molecule and further components by hydrophobic interaction chromatography comprising applying the solution comprising the molecule to an oppositely charged stationary phase, and eluting the molecule from the stationary phase in a mobile phase, characterised in that the stationary phase comprises a charged amphipathic sugar polymer(s).

A charged stationary phase that comprises a charged amphipathic sugar polymer(s) may take the form of a charged amphipathic sugar polymer(s) immobilised within a support (e.g. a polyacrylamide gel crosslinked with a cyclodextrin or an inulin) or immobilised on a support. Suitable supports include agarose, acrylate, cellulose, dextrin, polystyrene, polyacrylamide, Sepharose™ and silica. The invention further provides a charged amphipathic sugar polymer immobilised on a support, e.g. agarose, acrylate, cellulose, dextrin, polystyrene, polyacrylamide, Sepharose™, silica. Alternatively, the charged stationary phase may consist of one or a mixture of charged amphipathic sugar polymer(s).

The charged stationary phase may be generated by temporary derivatisation of an ion exchange matrix, suitably by contacting a solution comprising the amphipathic sugar polymer(s) with the ion exchange matrix, e.g. by running a solution comprising the amphipathic sugar polymer(s) through an ion exchange column. Suitable concentrations of amphipathic sugar polymers for derivatisation of ion exchange matrices are from 0.01 to 500 mg sugar polymer/ml matrix, preferably from 0.1 to 50 mg sugar polymer/ml matrix, most preferably from 1 to 30 mg sugar polymerlml matrix.

The sugar polymers used in methods of the invention must have an amphipathic nature (both hydrophilic and hydrophobic surfaces).

A mixture of amphipathic sugar polymers may be a mixture of positively charged amphipathic sugar polymers, or a mixture of negatively charged amphipathic sugar polymers. Alternatively the mixture of amphipathic sugar polymers can be a mixture of positively and negatively charged sugar polymers which mixture itself will be positively or negatively charged depending on the relative proportions and charge of the charged species.

An amphipathic sugar polymer useful in a method of the invention can be a cyclic sugar polymer, preferably a glucosan or a derivative thereof, more preferably a cyclodextrin or derivative thereof, most preferably an α-cyclodextrin, a β-cyclodextrin, a γ-cyclodextrin, or a derivative thereof. β-cyclodextrin and derivatives thereof are particularly favoured. Suitable cyclodextrin derivatives for use in methods of the invention include negatively charged derivatives such as cyclodextrin sulfate, sulfopropyl cyclodextrin, sulfobutylether cyclodextrin, cyclodextrin phosphate, carboxymethyl cyclodextrin, carboxyethyl cyclodextrin and succinyl hydroxypropyl cyclodextrin; and positively charged derivatives such as quartemary ammonium cyclodextrin and 6-monodeoxy-6-monoamino cyclodextrin.

An amphipathic sugar polymer useful in a method of the invention can be a helical or a linear sugar polymer.

An amphipathic sugar polymer useful in a method of the invention can be a fructosan or a derivative thereof, more preferably an inulin or a derivative thereof. Suitably the inulin or inulin derivative has a degree of polymerisation (DP) of from about 3 to 500, 3 to 250 or 3 to 100, preferably from 3 to 50, more preferably from 10 to 50, yet more preferably from 15 to 40, further preferably from 20 to 30, e.g. 20, 21, 22, 23, 24, 25, 26, 27, 28, 29, 30.

Charged inulin derivatives suitable for use in a method of the invention carry a net positive or negative charge at the pH at which the method is performed.

Charged inulin derivatives can be derivatised by one or more type(s) of charged group, e.g. selected from the group consisting of: a sulfonyl group, a sulfonylalkyl group, a phosphonyl group, a phosphonylalkyl group, a carboxy group, a carboxyalkyl group, an alkyl-succinyl group, a quaternary ammonium group, an aminoalkyl group, an amino group, an alkylamino group and a dialkylamino group.

The charged inulin derivatives may also be derivatised by one or more type(s) of non-polar hydrocarbyl group, e.g. those selected from the group comprising a linear alkyl derivative(s), branched alkyl derivative(s) or a mixture of linear alkyl derivative(s) and branched alkyl derivative(s).

Suitable charged inulin derivatives for use in a method of the invention include: negatively charged inulins such as sulfonated inulin, carboxmethyl inulin, carboxethyl inulin, alkyl-succinyl-inulin (e.g. octyl-succinyl-inulin), and positively charged inulins such as quartemary ammonium inulin, and aminoalkylinulin (e.g, aminopropyl inulin).

A preferred inulin derivative for use in a method of the invention Is a compound of formula (I):

G(O—CO—NH—R¹)ₐ-[F(O—CO—NH—R²)_{b}]ₙ (I)

wherein:
G is a terminal glucosyl unit in which one or more hydroxyl groups thereof may be substituted with a group or groups of formula (O—CO—NH—R¹);
R¹ Is a charged substituent or a straight or branched chain saturated or unsaturated hydrocarbyl group having from 1 to 25 carbon atoms, said saturated or unsaturated hydrocarbyl group optionally being substituted with one or more charged substituents, and, where there is more than one (O—CO—NH—R¹) group on the glucosyl unit, each R¹ group may be the same or different;
a is an integer of from 0 to 4;
F is a fructosyl unit in which one or more hydroxyl groups thereof may be substituted with a group or groups of formula (O—CO—NH—R²);
R² is a charged substituent or a straight or branched chain saturated or unsaturated hydrocarbyl group having from 1 to 25 carbon atoms, said saturated or unsaturated hydrocarbyl group optionally being substituted with one or more charged substituents, and, where there is more than one (O—CO—NH—R²) group on the fructosyl unit, each R² group may be the same or different;
b is an integer of from 0 to 3 and from 0 to 4 for the terminal fructosyl unit;
n is an integer of from 2 to 499 preferably of from 2 to 249, more preferably 2 to 99, yet more preferably 2 to 49, further preferably 9 to 49, yet further preferably 14 to 39, more preferably of from 19 to 29, e.g. 19, 20, 21, 22, 23, 24, 25, 26, 27, 28, or 29, most preferably n is 24,
each unit of formula F(O—CO—NH—R²)b may be the same or different from any other unit of formula F(O—CO—NH—R²)_{b}; and
the average degree of substitution per glucosyl or fructosyl unit is from 0.02 to 3.0.

Each group R¹ and R² may be selected from alkyl, alkenyl and alkynyl groups having from 1 to 25, preferably 3 to 22, most preferably 3 to 18 carbon atoms. One or more of the groups R¹ and R² can be an alkyl group having from 1 to 25, preferably 3 to 22, most preferably 3 to 18 carbon atoms; suitably one or more of groups R¹ and R² is an alkenyl or alkynyl group having from 1 to 25, preferably 3 to 22, most preferably 3 to 18 carbon atoms. Each alkyl group R¹ and R² can be a linear alkyl group having from 1 to 25, preferably 3 to 22, most preferably 3 to 18 carbons or branched alkyl group having from 3 to 25, preferably 3 to 22, most preferably 3 to 18 carbons.

The compound of formula (I) can be a polydisperse linear or slightly branched inulin N-alkylurethane, e.g. selected from the group consisting of inulin N-n-octyl-carbamates, inulin N-n-dodecylcarbamates and inulin N-n-octadecylcarbamates.

Where R¹ is a hydrocarbyl group substituted with a charged substitutent, said charged substitutent is preferably selected from the group consisting of: a sulfonyl group, a phosphonyl group, a carboxy group, an alkyl-succinyl group, a quaternary ammonium group and an amino group.

Where R² is a hydrocarbyl group substituted with a charged substitutent, said charged substitutent is preferably selected from the group consisting of: a sulfonyl group, a phosphonyl group, a carboxy group, an alkyl-succinyl group, a quaternary ammonium group and an amino group.

Where R¹ and/or R² is a charged substitutent, said charged substitutent is preferably selected from the group consisting of: a sulfonyl group, a phosphonyl group, a carboxy group, an alkyl-succinyl group, a quaternary ammonium group and an amino group.

A charged inulin derivative according to formula (I) has a net positive or negative charge at the pH at which the method is performed.

The average degree of substitution per glucosyl or fructosyl unit is suitably from 0.02 to 3.0, preferably from 0.05 to 1.0, most preferably from 0.05 to 0.5.

The ratio of charged side chains to non-charged alkyl side chains is suitably in the range 1:10 to 10:1, preferably around 1:1. For a charged inulin of formula (I), generally at least one charged derivatisation and one alkyl derivatisation will be present on the inulin.

Where the inulin chain length (DP) is in the range from 20 to 30 molecules (e.g. around 25 sugar units). The DS (derivatisation ratio) is typically around 0.2. Examples of inulins useful in methods of the invention include, but are not limited to, those with 50% of the derivatisations being charged side chains and 50% being alkyl derivatisations.

The term amphipathic sugar polymer(s) encompasses a single type of amphipathic sugar polymer or a mixture of amphipathic sugar polymers.

In some instances, the eluate collected after separation containing the separated molecule may contain amphipathic polymer(s). If desired, the amphipathic polymer(s) can be removed from the eluate, and thus from the molecule. Methods for removal of amphipathic polymer(s) from the eluate include dialysis, diafiltration, and/or chromatography. The amphipathic sugar polymer(s) can be removed by degradation of the amphipathic sugar polymer(s), which can be performed by one or more of the following methods: chemical degradation, enzymic digestion, electromagnetic radiation, shear stress, or heat. Preferably degradation is by enzymic digestion, more preferably degradation is by glucosyltransferase, amylase, xylanase digestion, exo-inulinase and/or endo-inulinase digestion. The choice of enzyme or enzymes for enzymic digestion is directed by the amphipathic sugar polymer or polymers employed. Enzymic degradation can be performed by contacting the eluate containing amphipathic polymer(s) with immobilised enzyme, for example by passing the eluate through a column containing immobilised enzyme. The sugar polymer can be degraded to its constituent monomers, the molecule of interest can then be purified using a method such as dialysis, diafiltration, and/or chromatography.

Separation methods of the invention can be used to separate, isolate and purify molecules that exhibit some degree of surface exposed hydrophobicity, i.e. molecules that are hydrophobic or amphiphilic. The methods are particularly useful to separate protein molecules, preferably proteins with one or more hydrophobic side chains, (e.g. a protein with a surface exposed hydrophobic amino acid, such as tryptophan). The term protein as used herein encompasses proteins, peptides, polypeptides and oligopeptides, enzymes, monomeric proteins and multimeric proteins which can be homomers or heteromers. Proteins may be synthetic or naturally occurring, and may be obtained by chemical synthesis, or by recombinant or non-recombinant methods. Separation methods of the invention are also particularly useful for separation, isolation and purification of a nucleic acid molecule, such as single or double stranded DNA or RNA or a DNA/RNA heteroduplex.

In a preferred separation method of the invention, the molecule of interest is a charged protein and is separated from further protein(s) present in the solution. The method is particularly useful for separation of a charged protein from further proteins in solution, especially from further similarly charged protein(s). In particular, purification of two proteins with differing hydrophobicities but very similar charge can be achieved using methods of the invention; such separations are currently extremely difficult or impossible to perform using existing ion exchange chromatography methods. Separations can sometimes be achieved using electrophoretic methods, but these generally require that the mobile phase be at an extreme pH, e.g. a very acidic pH in the range pH 2 to pH 5. These aggressive low pH conditions may cause degradation or otherwise affect the molecule of interest. If the molecule of interest is a protein, it is liable to be denatured at low pH and inactivated. Thus after separation by electrophoretic methods it may be necessary to attempt to renature the protein, which may not be successful and will result in loss of yield of native, functional protein. Methods of the invention are advantageous as they can be used to achieve effective separation of molecules, in particular molecules such as proteins and nucleic acids, at pH in the range 5 to 9, so it is possible to perform the separation methods using reaction conditions that do not result in significant degradation of, or damage to, the molecule of interest.

Where a protein or proteins are to be separated using a method of the invention the interaction between amphipathic sugar polymers and hydrophobic amino acid side chains of the protein(s) of interest can be modulated. The amphipathic sugar polymer—protein interaction can be used to increase or decrease the affinity of the protein for the chromatography matrix. Under such conditions the protein—matrix interaction will be determined by the hydrophobic protein—sugar polymer interactions, in addition to or instead of the charge based protein—matrix interactions. In free solution, binding of the charged amphipathic sugar polymer to the protein changes the effective net charge of the protein, altering its behaviour during ion exchange purification. Binding of the charged amphipathic sugar polymer to the charged stationary phase (ion exchange chromatography matrix) allows the matrix to interact with the protein through charge and/or hydrophobic effects. Therefore, methods of the invention can solve the problem of separating similarly charged proteins using ion exchange chromatography.

### LIST OF FIGURES

In each of the figures conductivity (mS/cm) and UV absorbance at 280 nm (mAu) of eluted fractions are plotted against time (min).

FIG. 1 shows the results when acetone in aqueous solution is run on a non-derivatised Q-Sepharose anion exchange column. Acetone is not retained on the column.

FIG. 2 shows the results when lysozyme in aqueous solution is run on a non-derivatised Q-Sepharose anion exchange column. Lysozyme is positively charged and is not retained on the positively charged column.

FIG. 3 shows the results when a mixture of acetone and lysozyme in aqueous solution is run on a non-derivatised 0-Sepharose anion exchange column. Neither the acetone, nor the lysozyme is retained on the column.

FIG. 4 shows the results when acetone in aqueous solution is run on a Q-Sepharose anion exchange column derivatised with sulfated beta cyclodextrin. Acetone is not retained on the derivatised column.

FIG. 5 shows the results when lysozyme in aqueous solution is run on a Q-Sepharose anion exchange column derivatised with sulfated beta cyclodextrin. Lysozyme is retained on the derivatised column and is eluted from the column (peak at 13 to 20 minutes) using a salt gradient.

FIG. 6 shows the results when a mixture of acetone and lysozyme in aqueous solution is run on a Q-Sepharose anion exchange column derivatised with sulfated beta cyclodextrin. Acetone is not retained on the column and is eluted from the column in under five minutes. Lysozyme is retained on the derivatised column and is eluted from the column (peak at 13 to 20 minutes) using a salt gradient. Using the derivatised column two UV absorbance peaks are seen confirming that the acetone and lysozyme have been separated on the derivatised column.

### EXAMPLES

The following methods are for the purification of basic proteins (high pI). Resin types (charge) would be reversed to separate acidic proteins.

### Example 1

### Method 1—Separation of a Strongly Hydrophobic Protein

A running buffer (mobile phase) is selected with a pH below the pI of the protein so that the protein carries a positive charge. Purification of the protein is achieved using a negatively charged sugar polymer carboxymethyl beta-cyclodextrin and a positively charged chromatography matrix (anion exchanger). Traditionally it would not be possible to retain a positively charged protein on a positively charged matrix. However, in this method, a solution of a negatively charged sugar polymer is injected through the column prior to introduction of the protein solution. The sugar polymer binds to the chromatography matrix, temporarily derivatising the surface. This derivatised surface is capable of hydrophobic interaction with protein molecules. The protein(s) to be separated are then injected through the column. An elution is performed. The elution method can be an isocratic elution (no salt gradient) in which retention time on the column is determined by the strength of the hydrophobic protein—sugar polymer interactions. A protein separation is achieved, the most hydrophobic protein is eluted last from the column. Thus a separation based on protein hydrophobicity is achieved using an ion-exchange matrix. Alternatively a salt gradient may be used for the elution. The elution may also involve the use of one or more other compounds (e.g. urea, methanol, ethanol, isopropyl alcohol, guanidine hydrochloride or acetonitrile) to modulate the hydrophobic interaction of the sugar polymer with protein.

### Chromatography Column:

| | |
|---|---|
| Type | Strong anion exchanger |
| Brand | Q sepharose fast flow |
| Manufacturer | Amersham |
| Size | 1 ml |

### Running Buffers:

| | |
|---|---|
| Tris | 100 mM |
| EDTA | 1 mM |
| pH | 8.0 |
| Flow | 1 ml/min |
| Salt gradient | NaCl 0-0.5 M |

### Target Protein:

| | |
|---|---|
| Type | Lysozyme (pl = 11.3) |
| Concentration | 0.5 mg/ml |
| Inject volume | 1 ml |

### Sugar Polymer:

| | |
|---|---|
| Description | Negatively charged, cyclodextrin derivative |
| Type | Sulfated-beta-cyclodextrin |
| Conc | 4 mg/ml |
| Inject | 25 ml |

A 1 ml Q Sepharose anion exchanger was employed to separate the protein lysozyme from acetone in aqueous solution. The process was operated as described in method 1 using a salt gradient to elute the protein and sugar polymer.

Lysozyme has a molecular weight of 14 kDa and an isoelectric point of 11.3. The running buffer used was 100 mM Tris, 1 mM EDTA at pH 8.0 (buffer 1). Therefore, both the protein and the chromatography stationary phase were positively charged under the process conditions. Charged molecules bound to the anion exchange resin were removed by elution using a salt gradient. This was performed by mixing buffer 1 with buffer 2 (as buffer 1 plus 1 M NaCl) in gradually increasing ratios of buffer 2.

The column was loaded with 1 ml samples for each of the six runs shown. The samples contained 0.25% v/v acetone, 0.5 mg/ml lysozyme or a mixture of 0.25% v/v acetone, 0.5 mg/ml lysozyme. All samples were dissolved in buffer 1. Each sample was run on the Q Sepharose column with a flow rate of 1 mil/min. The experiments were performed by first running each of the acetone, lysozyme and mixed acetonellysozyme samples individually down the underivatised column. This process was repeated using the same column, but using a derivatisation step to derivatise the column with sulfated-beta-cyclodextrin before running each sample down the column. To derivatise the column the sulphated beta-cyclodextrin was loaded onto the column by injecting 25 ml of buffer 1 containing 4 mg/ml of the sulphated beta-cyclodextrin at a flow rate of 4 ml/min. The sugar polymer used, sulphated beta-cyclodextrin sodium salt (Aldrich, #38,915-3), is negatively charged.

Elution of acetone and lysozyme was monitored continuously by detecting UV absorbance at 280 nm (mAu) of the elute. Lysozyme and acetone absorb UV light at 280 nm. Sulphated beta-cyclodextrin does not absorb UV light at 280 nm sufficiently strongly to affect the UV signals measured. Conductivity was measured in mS/cm. The conductivity of buffer 1 was around 8 mS/cm and of buffer 2 was around 80 mS/cm.

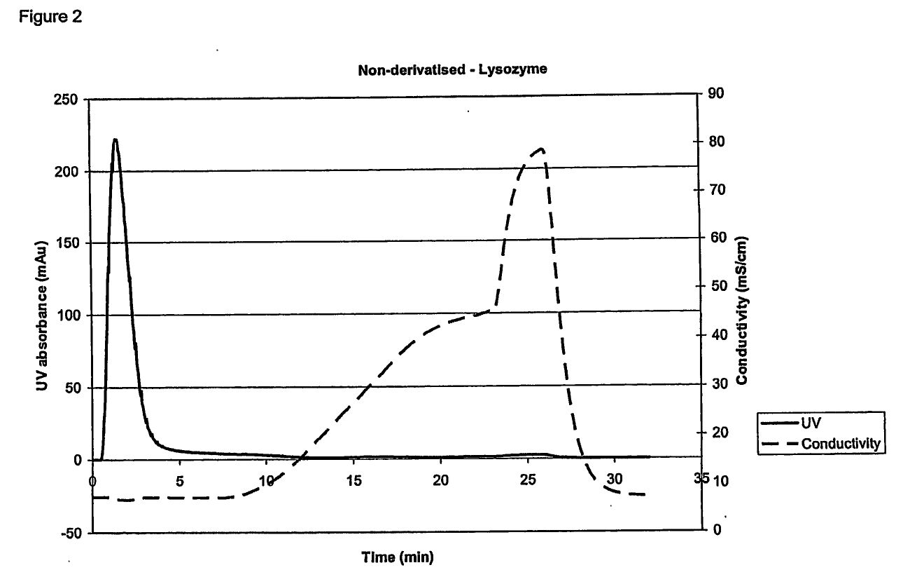

Under normal operation, (i.e. without derivatisation of the anion exchange resin), lysozyme cannot be retained on an anion exchange column as both the column and protein have the same charge. Since the flow rate is 1 ml/mn and the column volume is 1 ml with around 50% voidage, material that cannot bind to the chromatography stationary phase begins to elute approximately 0.5 min after injection (FIG. 2).

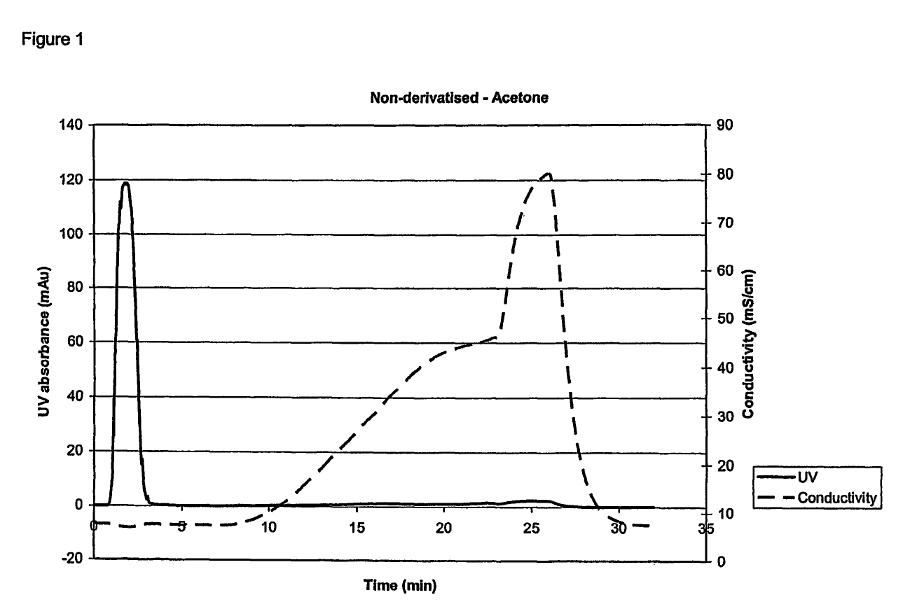

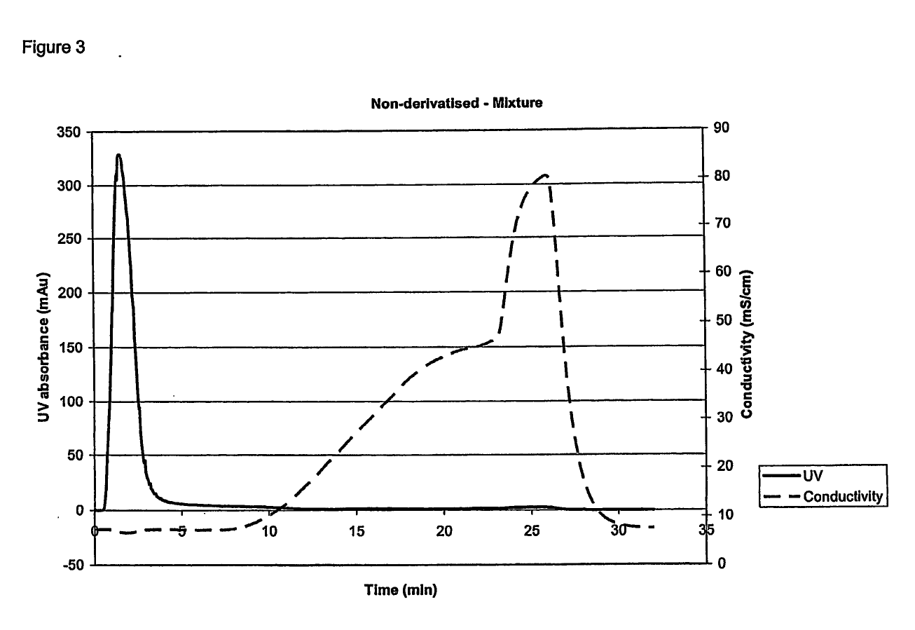

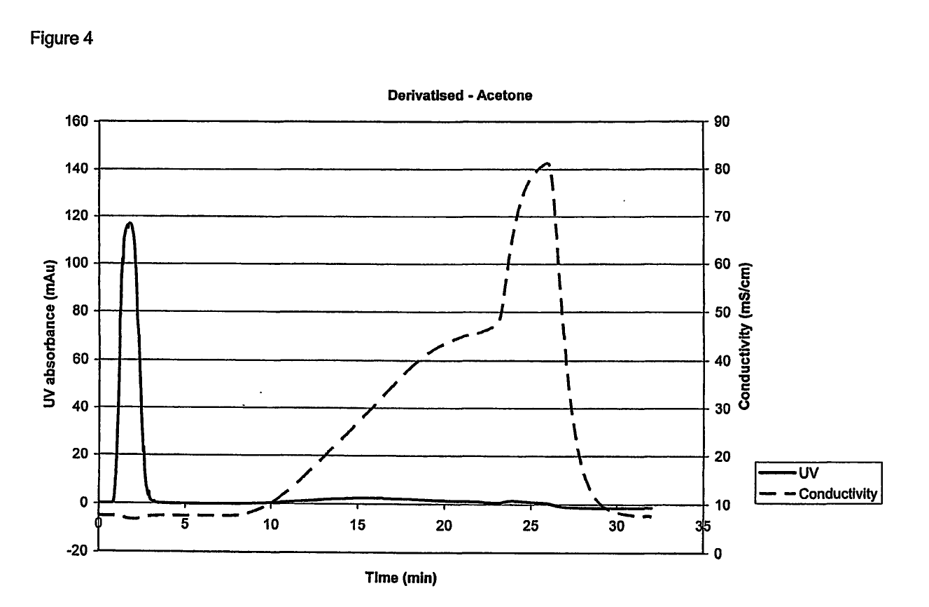

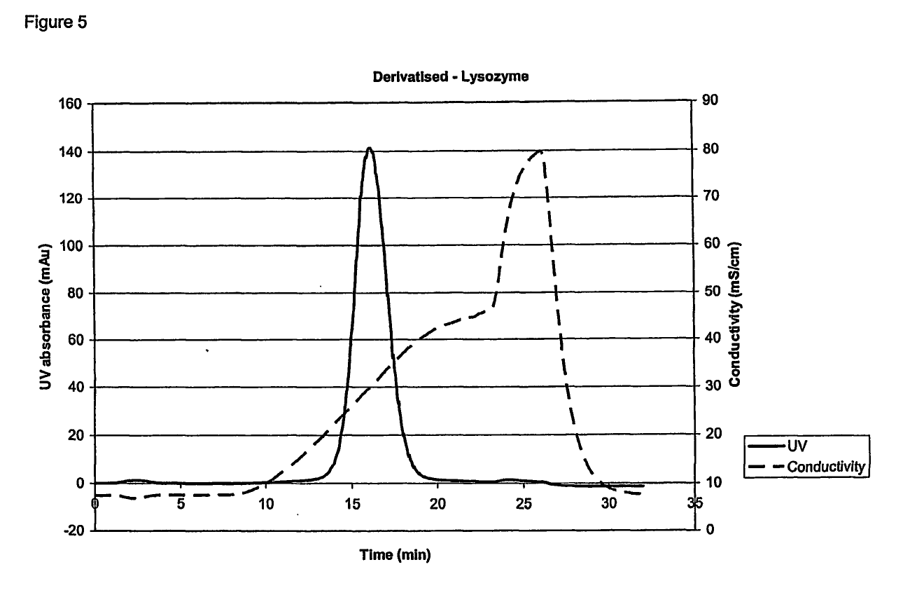

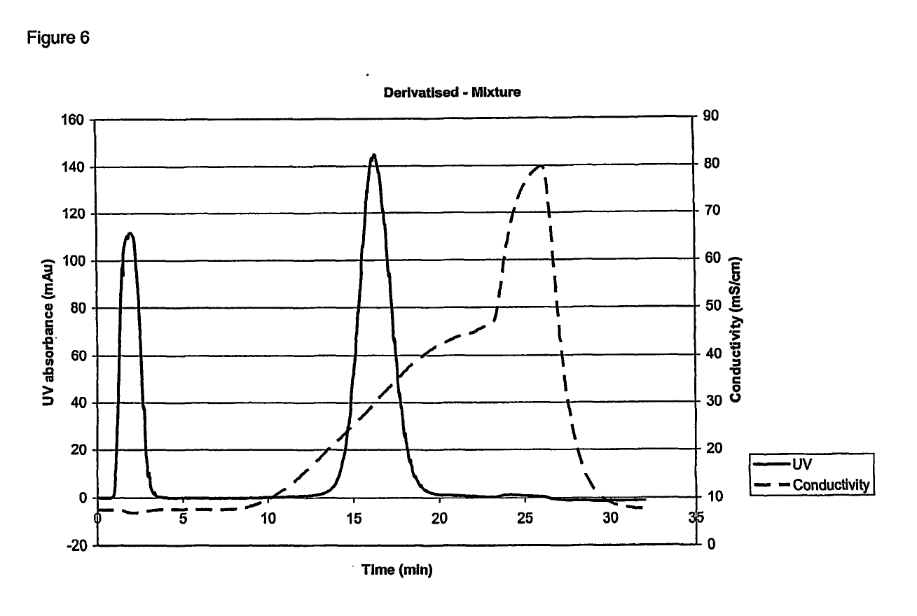

TABLE 1

| | | Sepharose anion exchange |
|---|---|---|
| | Sepharose anion exchange | column - Derivatised with |
| Sample | column - Non-derivatised | sulphated beta-cyclodextrin |
| Acetone | No retention (FIG. 1) | No retention (FIG. 4) |
| Lysozyme | No retention (FIG. 2) | Retained (FIG. 5) |
| Mixture of | No retention (FIG. 3) | Separated (FIG. 6) |
| Acetone and | | |
| Lysozyme | | |

All three samples run on the underivatised column eluted rapidly without binding to the column (FIGS. 1 to 3). The same process occurred when acetone was injected onto the sulphated beta-cyclodextrin-derivatised column (FIG. 4). Acetone is uncharged and thus does not interact with the anion exchange resin; acetone forms no hydrophobic interaction with cyclodextrin sugar polymer and so elutes without binding to either the non-derivatised or derivatised column.

When the lysozyme sample was injected onto the derivatised column, no increase in UV absorbance was detected in the 0 to 5 minute range, as the protein was retained on the derivatised column (FIG. 5). When a salt gradient was run down the column, the UV absorbance Increased rapidly, indicating that the retained protein was being eluted from the column (FIG. 5).

In the final run, a sample containing a mixture of acetone and lysozyme was found to be separated efficiently on the derivatised column. The acetone passed through the column, but the lysozyme protein was retained until it was eluted from the column using a salt gradient. These results demonstrate that charged amphipathic sugar polymer—protein interactions can be used in ion-exchange chromatography to achieve separation.

### Method 2—Separation of a Weakly Hydrophobic Protein

In this method a positively charged amphipathic sugar polymer (e.g. an amino beta-cyclodextrin or an aminoalkyl inulin) and a negatively charged chromatography matrix (cation exchanger) are used. The column is derivatised with a pulse of the positively charged sugar polymer in aqueous solution. A solution containing the positively charged weakly hydrophobic protein is then injected through the column. This method is mixed mode, that is protein—matrix interactions occur through a combination of electrostatic and hydrophobic interactions. Elution is performed using a gradually increasing salt concentration to screen out the protein—matrix electrostatic interactions. However, since the protein is bound through a combination of electrostatic and hydrophobic interactions elution of the more hydrophobic protein will occur at a higher salt concentration. This method is particularly useful for separation of two proteins with very similar charge using an ion exchange matrix.

### Chromatography Column:

| | |
|---|---|
| Type | Weak cation exchanger |
| Brand | CM sepharose fast flow |
| Manufacturer | Amersham |
| Size | 1 ml |

### Running Buffers:

| | |
|---|---|
| Tris | 100 mM |
| EDTA | 1 mM |
| pH | 8.0 |
| Flow | 1 ml/min |
| Salt gradient | NaCl 0-0.5 M |

### Protein:

| | |
|---|---|
| Type | Lysozyme (pl = 11.3) with |
| | Cytochrome c (pl = 10.6), alpha chymotrypsinogen |
| | (pl = 9.5) or ribonuclease A (pl = 9.6) |
| Concentration | 0.5 mg/ml |
| Inject volume | 1 ml |

### Sugar Polymer:

| | |
|---|---|
| Description | Weakly positively charged, cyclodextrin derivative |
| Type | 6-monodeoxy-6-monoamino-beta-cyclodextrin |
| | hydrochloride |
| or | |
| Description | Weakly positively charged, inulin derivative |
| Type | aminopropyl inulin |
| Conc | 4 mg/ml |
| Inject | 25 ml |

### Method 3

In this method, the hydrophobic interaction between the amphipathic sugar polymer and the positively charged protein of interest is used to decrease binding to the chromatography matrix. The sugar polymer used has a weak negative charge (e.g. carboxymethyl beta-cyclodextrin). A negatively charged chromatography matrix (cation exchanger) is used to ensure that there is little or no binding of the sugar polymer to the matrix. The positively charged protein is passed through the column and binds to the negatively charged matrix. The sugar polymer is now included in the elution buffer and a salt gradient passed through the column. The sugar polymer binds to the most hydrophobic protein with greatest affinity. This reduces the protein net charge and allows the most hydrophobic protein to be eluted at a lower salt concentration than a similarly charged less hydrophobic protein.

### Method 4—Optional Step—Removal of Amphipathic Sugar Polymer(s)

Eluent obtained from the ion exchange column is passed through a second column, or column section, containing an immobilised enzyme. The enzyme degrades any sugar polymer that co-elutes with the protein(s) of interest. Sugar polymers are degraded to monomers, e.g. glucose, fructose and/or derivatives thereof. This releases the sugar polymers from the protein molecules and prevents any further modification of the protein properties by the sugar polymer.

1. A method for chromatographic separation of a molecule, wherein a mobile phase and charged stationary phase are present and a charged amphipathic sugar polymer(s) is employed to modify the hydrophobic interaction between said molecule and said charged stationary phase, and wherein the pH of said mobile phase is below the pI of the molecule (and thus the molecule carries a net positive charge) or the pH of the mobile phase is above the pI of the molecule (and thus the molecule carries a net negative charge).

2-4. (canceled)

5. The method of claim 1 for separating a positively charged molecule from a solution comprising said the molecule and further components by hydrophobic interaction chromatography comprising applying a solution comprising said molecule to a positively charged stationary phase which is non-covalently associated with a negatively charged amphipathic sugar polymer(s), and eluting said molecule from said stationary phase in a mobile phase.

6. The method of claim 1 for separating a negatively charged molecule from a solution comprising said he molecule and further components by hydrophobic interaction chromatography comprising applying a solution comprising said molecule to a negatively charged stationary phase which is non-covalently associated with a positively charged amphipathic sugar polymer(s), and eluting said molecule from said the stationary phase in a mobile phase.

7. The method of claim 1 for separating a positively charged molecule from a solution comprising said molecule and further components by mixed mode hydrophobic interaction/ion exchange chromatography comprising applying a solution comprising said molecule to a negatively charged stationary phase which is non-covalently associated with a positively charged amphipathic sugar polymer(s), and eluting said the molecule from said the stationary phase in a mobile phase.

8. The method of claim 1 for separating a negatively charged molecule from a solution comprising the molecule and further components by mixed mode hydrophobic interaction/ion exchange chromatography comprising applying a solution comprising the molecule to a positively charged stationary phase which is non-covalently associated with a negatively charged amphipathic sugar polymer(s), and eluting the molecule from the stationary phase in a mobile phase.

9. The method of claim 1, wherein the mobile phase comprises an amphipathic sugar polymer(s).

10. The method of claim 1, wherein the mobile phase comprises a charged amphipathic sugar polymer(s).

11. The method of claim 1 for separating a positively charged molecule from a solution comprising the molecule and further components by mixed mode hydrophobic interaction/ion exchange chromatography comprising applying a solution comprising the molecule to a negatively charged stationary phase, and eluting the molecule from the stationary phase in a mobile phase comprising a negatively charged amphipathic sugar polymer(s).

12. The method of claim 1 for separating a negatively charged molecule from a solution comprising the molecule and further components by mixed mode hydrophobic interaction/ion exchange chromatography comprising applying a solution comprising the molecule to a positively charged stationary phase, and eluting the molecule from the stationary phase in a mobile phase comprising a positively charged amphipathic sugar polymer.

13-49. (canceled)

50. A method for separating a molecule from a solution comprising a charged molecule and further components by hydrophobic interaction chromatography comprising applying the solution comprising the molecule to an oppositely charged stationary phase, and eluting the molecule from the stationary phase in a mobile phase, characterised in that the stationary phase comprises a charged amphipathic sugar polymer(s).

51. A method for separating a molecule from a solution comprising the molecule and further components by hydrophobic interaction chromatography comprising applying the solution comprising the molecule to a charged stationary phase, and eluting the molecule from the stationary phase in a mobile phase, characterised in that:
(a) the charged stationary phase is non-covalently associated with a charged amphipathic sugar polymer(s), or the stationary phase comprises a charged amphipathic sugar polymer, and/or
(b) the molecule is non-covalently associated with a charged amphipathic sugar polymer(s),
and wherein the pH of the mobile phase is below the pI of the molecule (and thus the molecule carries a net positive charge) or the pH of the mobile phase is above the pI of the molecule (and thus the molecule carries a net negative charge).

52. A method according to claim 51 for separating a positively charged molecule from a solution comprising the molecule and further components by hydrophobic interaction chromatography comprising applying a solution comprising the molecule to a positively charged stationary phase which is non-covalently associated with a negatively charged amphipathic sugar polymer(s), and eluting the molecule from the stationary phase in a mobile phase.

53. A method according to claim 51 for separating a negatively charged molecule from a solution comprising the molecule and further components by hydrophobic interaction chromatography comprising applying a solution comprising the molecule to a negatively charged stationary phase which is non-covalently associated with a positively charged amphipathic sugar polymer(s), and eluting the molecule from the stationary phase in a mobile phase.

54. A method according to claim 51 for separating a positively charged molecule from a solution comprising the molecule and further components by mixed mode hydrophobic interaction/ion exchange chromatography comprising applying a solution comprising the molecule to a negatively charged stationary phase which is non-covalently associated with a positively charged amphipathic sugar polymer(s), and eluting the molecule from the stationary phase in a mobile phase.

55. A method according to claim 51 for separating a negatively charged molecule from a solution comprising the molecule and further components by mixed mode hydrophobic interaction/ion exchange chromatography comprising applying a solution comprising the molecule to a positively charged stationary phase which is non-covalently associated with a negatively charged amphipathic sugar polymer(s), and eluting the molecule from the stationary phase in a mobile phase.

56. A method according to claim 51, wherein the mobile phase comprises an amphipathic sugar polymer(s).

57. A method according to claim 51, wherein the mobile phase comprises a charged amphipathic sugar polymer(s).

58. A method according to claim 51 for separating a positively charged molecule from a solution comprising the molecule and further components by mixed mode hydrophobic interaction/ion exchange chromatography comprising applying a solution comprising the molecule to a negatively charged stationary phase, and eluting the molecule from the stationary phase in a mobile phase comprising a negatively charged amphipathic sugar polymer(s).

59. A method according to claim 51 for separating a negatively charged molecule from a solution comprising the molecule and further components by mixed mode hydrophobic interaction/ion exchange chromatography comprising applying a solution comprising the molecule to a positively charged stationary phase, and eluting the molecule from the stationary phase in a mobile phase comprising a positively charged amphipathic sugar polymer.

60. A method according to claim 1 or claim 51, wherein the or an amphipathic sugar polymer is selected from the group consiting of a cyclic helical or linear sugar polymer.

61. A method according to claim 1 or claim 51, wherein the or an amphipathic sugar polymer is a cyclodextrin or derivative thereof.

62. A method according to claim 1 or claim 51, wherein the amphipathic sugar polymer is an inulin or a derivative thereof.

63. A method according to claim 1 or claim 51, wherein the amphipatic sugar polymer is an inulin derivative is substituted with one or more charged group selected from the group consisting of: a sulfonyl group, sulfonylalkyl group, a phosphonyl group, a phosphonylalkyl group, a carboxyl group, a carboxyalkyl group, an alkyl-succinyl group, a quaternary ammonium group, an aminoalkyl group, an amino group, an alkylamino group and a dialkylamino group

64. A method according to claim 1 or claim 51 further comprising removal of amphipathic sugar polymer(s) from the eluate.

65. A method according to claim 1 or claim 51, wherein the molecule for separation is a protein or a nucleic acid. _
